(19) United States
(12) Patent Application Publication
(10) Pub. No.: US 2016/0201143 A1
(43) Pub. Date: Jul. 14, 2016

(54) LKB1 Levels and Brain Metastasis from Non-Small-Cell Lung Cancer (NSCLC)

(71) Applicant: The University of North Carolina at Chapel Hill, Chapel Hill, NC (US)

(72) Inventors: David Neil Hayes, Chapel Hill, NC (US); Ni Zhao, Carrboro, NC (US)

(21) Appl. No.: 15/073,832

(22) Filed: Mar. 18, 2016

Related U.S. Application Data
(63) Continuation of application No. 13/863,842, filed on Apr. 16, 2013.
(60) Provisional application No. 61/624,797, filed on Apr. 16, 2012.
(60) Provisional application No. 61/793,060, filed on Mar. 15, 2013.

(51) Int. Cl. C12Q 1/68

(52) U.S. Cl. CPC C12Q 1/6886; C12Q 2600/156; C12Q 2600/158; C12Q 2600/112

(57) ABSTRACT

This invention is directed to a method to increased likelihood of brain metastasis from NSCLC in a subject by measuring the levels of LKB1. The invention also provides related kits.

### CROSS REFERENCE TO RELATED APPLICATIONS

This application claims the benefit of U.S. Provisional Appn. 61/793,060 filed Mar. 15, 2013, Hayes et al. entitled “LKB1 Levels and Brain Metastasis from Non-Small-Cell Lung Cancer (NSCLC)” having Atty. Dkt. No. UNC13002usv2 and U.S. Provisional Appn. 61/624,797 filed Apr. 16, 2012, Hayes, entitled “Method of Prognosing the Risk of Brain Metastasis from Lung Cancel” having Atty. Docket No. UNC-0001, the contents of which are hereby incorporated by reference in their entirety.

### 1. FIELD OF THE INVENTION

This invention is directed to a method to determine the likelihood of brain metastasis from non-small-cell lung cancer (NSCLC) using either LKB1 or a combination of LKB1 and KRAS.

### 2. BACKGROUND OF THE INVENTION

2.1. Introduction

Non-small-cell lung cancer (NSCLC) is the leading cause of cancer-related deaths in the United States with brain metastasis as one of the most malicious complications, which leads to very high morbidity and mortality ^{1,2}. Historically, the prognosis of NSCLC with brain metastasis has been poor, with a median overall survival of 4.5 months for patients treated with standard whole brain radiation therapy (WBRT) and 4-11 weeks in untreated patients ³. The prevalence of brain metastasis in NSCLC is reported to be increasing, possibly due to improved diagnosis in brain imaging and prolonged survival with new systemic treatment options. Therefore, identification of biomarkers that have critical roles in cell growth, metabolism, and tumor recurrence would provide valuable information in disease prognosis and better treatment choices.

In the past few years, several lines of evidence implicate the importance of serine-threonine kinase liver kinase B1 (LKB1, also known as STK11) as a tumor suppressor gene in lung cancer development and progression in both human and model organisms ⁴⁻⁶. LKB1 was first identified in 1997 as the causative mutation in the autosomal-dominant inherited Peutz-Jeghers syndrome (PJS), which is characterized by mucocutaneous pigmentation and multiple benign polyps (hamartomas) and increased risk for gastrointestinal and extraintestinal malignancies ⁷. LKB1 loss is one of the most frequent genetic alterations in NSCLC⁴, the inactivation of which has also been proposed to be associated with tumor metastasis in lung cancer and other tumor types ^{5,6,8,9}. Specifically, LKB1 mutation or loss of heterozygosity (LOH) of 19p13.2 which harbors the LKB1 gene was observed in a much higher proportion in brain metastases of lung cancer patients than in the primary tumors ^{5,8}.

However, the mechanism through which LKB1 is inactivated remains a puzzle, with potential mechanisms including homozygous deletion, point mutations and epigenetic silencing ^{5,6}. The discrepancy between the high frequency of LOH (often over 50%) of 19p13.3 ⁸, and the reported rate of LKB1 mutation ^{4,5} suggests a number of questions, including ones regarding the accuracy of reported gene mutation studies and an alternative mechanism of LKB1 silencing. Epigenetic events like promoter methylation, heterozygous or homozygous deletion and mutation are the three most important ways of gene silencing. Integrated analysis incorporating these measurements can shed lights on the mechanism of LKB1's effect of tumorigenesis as well as metastasis.

With regard to reference 8, Sobottka et al., they found LKB1 alterations in tissues taken from actual brain metastasis. Such alterations may have arisen after metastasis or as part of the progression of the tumor in the brain. Furthermore, they examined microsatellite instability. While microsatellite instability may be associated with copy number loss, it is also associated with copy neutral states or even amplification.

### 3. SUMMARY OF THE INVENTION

This invention is directed to a method for determining the likelihood of brain metastasis from non-small-cell lung cancer (NSCLC) which comprises: measuring a level of LKB1 in a sample from the subject; and if the level of LKB1 is reduced than a normal level, determining that the subject has increased likelihood of brain metastasis. The level of LKB1 may be measured by copy number or a mass spectrometry assay. The sample may be a formalin-fixed, paraffin-embedded sample, a fresh-frozen sample or a fresh sample.

In one embodiment, the level of KRAS is also measured.

A method of selecting non-small-cell lung cancer (NSCLC) patients for prophylactic cranial irradiation (PCI) which comprises measuring the level of LKB1 in a patient sample and if the levels are reduced selecting the patient for prophylactic cranial irradiation (PCI). The method of claim 10, further comprising measuring the level or mutations in KRAS.

The invention also includes a kit comprising: at least one reagent selected from the group consisting of: a nucleic acid probe capable of specifically hybridizing with an mRNA encoding LKB1; a pair of nucleic acid primers capable of PCR amplification of an mRNA encoding LKB1; and instructions for use in measuring a level of LKB1 in a proliferative phase sample from a subject suspected of having increased likelihood of brain metastasis from NSCLC wherein levels of LKB1 at least 2-fold or greater are indicative of increased likelihood of brain metastasis from NSCLC in the subject.

### 4. BRIEF DESCRIPTION OF THE FIGURES

FIG. 1A-1C shows that different measurements of LKB1 genetic alterations are correlated (wild type black dots, mutant gray dots). FIG. 1A: LKB1 wild type group has significantly higher gene expression. FIG. 1B: LKB1 expression and copy number are positively correlated. FIG. 1C: wild type group has significantly higher copy number.

FIG. 2A-2C shows different measurements of KRAS genetic alteration are correlated (wild type black dots, mutant gray dots), FIG. 2A: KRAS wild type samples had a significantly lower gene expression. FIG. 2B: KRAS expression and copy number are positively correlated. FIG. 2C: wild type group has significantly lower copy number.

FIG. 3: ROC curve for the multivariant predictive model. Predictors in this model include LKB1 copy number, Kras mutation, patients' age at diagnosis and nodal stage. P values were generated by testing the hypothesis that area under the curve (AUC) is 0.5.

### 5. DETAILED DESCRIPTION OF THE INVENTION

5.1. Background

Brain metastasis is one of the most malicious complications of non-small-cell lung carcinoma (NSCLC) and is associated with extremely high morbidity and mortality. Promising treatment strategies have been suggested that can prolong patients' survival in NSCLC with brain metastasis. Therefore, genetic markers that are associated with brain metastasis will be very helpful in patient management. Recent years of investigation suggested a role of serine-threonine kinase liver kinase B1 (LKB1, a.k.a. STK11) in NSCLC development and progression in both human and model organisms, in synergy with KRAS alteration. In this study, we systematically analyzed how LKB1 and KRAS alteration, measured by mutation, gene expression (GE) and copy number (CN) alteration, are associated with brain metastasis in NSCLC patients.

The predictive model, although not perfect, can be useful in selecting patients at higher risk of brain metastasis and eligible for corresponding prevention and treatment in clinical setting. Specifically, lower LKB1 copy number and KRAS mutation were significantly associated with more lung cancer brain metastasis. Patients with higher LKB1 copy number or wild type KRAS had lower risk of developing brain recurrence during the lung cancer follow up. Patients with lower LKB1 copy number or mutant KRAS had higher risk of developing brain recurrence during the lung cancer follow up. LKB1 copy number and KRAS status can be used as a preliminary genetic marker to predict brain metastasis in NSCLC and to guide treatment.

A method for determining the brain metastasis potential of a lung cancer in a mammal, said method comprising determining, in a tissue sample from said mammal, the presence or absence of LKB1 and correlating said presence or absence with said brain metastasis potential. The tissue sample may be a tumor biopsy. The absence of said LKB1 or decrease of the activity of said LKB1 indicates said lung cancer is likely to metastasize to the brain. Moreover, the presence of said intact LKB1 indicates said lung cancer is not likely to metastasize to the brain. The method may further comprise determining the presence or absence of a mutation in K-ras and the presence of wild type K-ras indicates said lung cancer is not likely to metastasize to the brain.

The sample may be a formalin-fixed, paraffin-embedded sample, needle biopsy, a fresh-frozen sample, a fresh sample, or a blood, cervical swab, uterine lavage, or urine sample. The tissue sample may be a tumor biopsy.

Moreover, the invention provides a kit comprising: (a) at least one reagent selected from the group consisting of: (i) a nucleic acid probe capable of specifically hybridizing with an mRNA encoding LKB1; (ii) a pair of nucleic acid primers capable of PCR amplification of an mRNA encoding LKB1; and (b) instructions for use in measuring a level of LKB1 in a sample from a subject with NSCLC wherein levels of LKB1 at least 2-fold or greater are indicative of greater likelihood of brain metastasis in the subject.

5.2. Definitions

The term “LKB1” as used herein refers to a nucleic acid encoding the LKB1 protein, also known as, EC 2.7.11.13, serine/threonine kinase 11 (SKT11) (Peutz-Jegheis syndrome); Entrez Gene: 67942, Ensembl: ENSG000001180467; OMIM: 6022165 UniProtKB: QI58313

The invention is directed to the measuring LKB1 levels including genes and proteins. The invention includes measuring, DNA comprising the entire or partial sequence of the nucleic acid sequence encoding LKB1, or the complement of such a sequence. The nucleic acids also include RNA comprising the entire or partial sequence of LKB1. A LKB1 protein may comprises the entire or partial amino acid sequence of any of the LKB1 protein or polypeptides. Fragments and variants of LKB1 genes and proteins are also encompassed by the present invention. By “fragment” is intended a portion of the polynucleotide or a portion of the amino acid sequence and hence protein encoded thereby. Polynucleotides that are fragment of the LKB1 nucleotide sequence generally comprise at least 10, 15, 20, 50, 75, 100, 150, 200, 250, 300, 350, 400, 450, 500, 550, 600, 650, 700, or 800, contiguous nucleotides, or up to the number of nucleotides present in a lull-length LKB1 polynucleotide disclosed herein. A fragment of a LKB1 polynucleotide will generally encode at least 15, 25, 30, 50, 100, 150, 200, or 250 contiguous amino acids, or up to the total number of amino acids present in a full-length LKB1 protein.

“Variant” is intended to mean substantially similar sequences. Generally, variants of a particular biomarker of the invention will have at least about 40%, 45%, 50%, 55%, 60%, 65%, 70%, 75%, 80%, 85%, 90%, 91 %, 92%, 93%, 94%, 95%, 96%, 97%, 98%, 99% or more sequence identity to that of LKB1 as determined by sequence alignment programs.

The invention is directed to measuring a LKB1 gene or protein whose level of expression in a tissue or cell is altered compared to that of a normal or healthy cell or tissue. As used herein, “underexpression” means expression greater than the expression detected in normal, healthy tissue. For example, an RNA transcript, copy number or its expression product that is underexpressed in a cell or tissue for a subject with increased likelihood of brain metastasis may be expressed at a level that is 25% times lower than in a in normal cell or tissue, such as 50% lower, 75% lower, 80% lower, 90% lower or less.

In some embodiments, expression, such as of a LKB1 copy number, RNA transcript or its expression product, is determined by normalization to the level of reference RNA transcripts or their expression products, which can be all measured transcripts (or their products) in the sample or a particular reference set of RNA transcripts (or their products). Normalization is performed to correct for or normalize away both differences in the amount of RNA assayed and variability in the quality of the RNA used. Therefore, an assay typically measures and incorporates the expression of certain normalizing genes, including well known housekeeping genes, such as, for example, GAPDH and/or β-Actin. Alternatively, normalization can be based on the mean or median signal of all of the assayed mRNAs or a large subset thereof (global normalization approach).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxy proline, gamma-carboxyglutamate, and O-phosphoserine. Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Primers” as used herein refer to oligonucleotides that can be used in an amplification method, such as a polymerase chain reaction (PCR), to amplify a nucleotide sequence based on the polynucleotide sequence corresponding to a particular genomic sequence, e.g., one specific for a particular CpG site. At least one of the PCR primers for amplification of a polynucleotide sequence is sequence-specific for the sequence.

The term “template” refers to any nucleic acid molecule that can be used for amplification in the technology. RNA or DNA that is not naturally double stranded can be made into double stranded DNA so as to be used as template DNA. Any double stranded DNA or preparation containing multiple, different double stranded DNA molecules can be used as template DNA to amplify a locus or loci of interest contained in the template DNA.

The term “amplification reaction” as used herein refers to a process for copying nucleic acid one or more times. In embodiments, the method of amplification includes, but is not limited to, polymerase chain reaction, self-sustained sequence reaction, ligase chain reaction, rapid amplification of cDNA ends, polymerase chain reaction and ligase chain reaction, Q-β replicase amplification, strand displacement amplification, rolling circle amplification, or splice overlap extension polymerase chain reaction. In some embodiments, a single molecule of nucleic acid may be amplified.

The term “sensitivity” as used herein refers to the number of true positives divided by the number of true positives plus the number of false negatives, where sensitivity (sens) may be within the range of 0<sens<1. Ideally, method embodiments herein have the number of false negatives equaling zero or close to equaling zero, so that no subject is wrongly identified as not having increased likelihood of brain metastasis from NSCLC when they indeed have increased likelihood of brain metastasis from NSCLC. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where sensitivity (spec) may be within the range of 0<spec<1. Ideally, the methods described herein have the number of false positives equaling zero or close to equaling zero, so that no subject is wrongly identified as having increased likelihood of brain metastasis from NSCLC when they do not in fact have increased likelihood of brain metastasis from NSCLC. Hence, a method that has both sensitivity and specificity equaling one, or 100%, is preferred.

5.3. Samples

The sample may be obtained using any of a number of methods in the art. A sample may be a sample of muscosal surfaces, a lung swab, a lung lavage sample, blood and blood fractions or products (e.g., serum, plasma, platelets, red blood cells, white blood cells, secreted protein or nucleic acids in the blood, free DNA isolated from blood, and the like), lymph, cultured cells, e.g., primary cultures, explants, and transformed cells, stool, urine, etc. The sample may also be vascular tissue or cells from blood vessels such as microdissected cells of lung origin. Examples of biological samples include those obtained from biopsies, such as punch biopsies, shave biopsies, fine needle aspirates (FNA), or surgical excisions; or biopsy from non-cutaneous tissues such as lymph node tissue, mucosa, or other embodiments. The biological sample may be a microdissected sample, such as a PALM-laser (Carl Zeiss Microimaging GmbH, Germany) capture microdissected sample.

A sample is typically obtained from a eukaryotic organism, most preferably a mammal such as a primate e.g., chimpanzee or human; cow; dog; cat; a rodent, e.g., guinea pig; rat; mouse; rabbit.

A sample can be treated with a fixative such as formaldehyde and embedded in paraffin (FFPE) and sectioned for use in the methods of the invention. Alternatively, fresh or frozen tissue may be used. These cells may be fixed, e.g., in alcoholic solutions such as 100% ethanol or 3:1 methanol:acetic acid. Nuclei can also be extracted from thick sections of paraffin-embedded specimens to reduce truncation artifacts and eliminate extraneous embedded material. Typically, biological samples, once obtained, are harvested and processed prior to hybridization using standard methods known in the art. Such processing typically includes protease treatment and additional fixation in an aldehyde solution such as formaldehyde.

5.3.1. Polynucleotide Sequence Amplification and Determination

In many instances, it is desirable to amplify a nucleic acid sequence encoding LKB1 using any of several nucleic acid amplification procedures which are well known in the art. Specifically, nucleic acid amplification is the chemical or enzymatic synthesis of nucleic acid copies which contain a sequence that is complementary to a nucleic acid sequence being amplified (template). The methods and kits of the invention may use any nucleic acid amplification or detection methods known to one skilled in the art, such as those described in U.S. Pat. No. 5,525,462 (Takarada et al.); U.S. Pat. No. 6,114,117 (Hepp et al.); U.S. Pat. No. 6,127,120 (Graham et al.); U.S. Pat. No. 6,344,317 (Urnovitz); U.S. Pat. No. 6,448,001 (Oku); U.S. Pat. No. 6,528,632 (Catanzariti et al.); and PCT Pub. No. WO 2005/111209 (Nakajima et al.); all of which are incorporated herein by reference in their entirety.

In some embodiments, the nucleic acids are amplified by PCR amplification using methodologies known to one skilled in the art. One skilled in the art will recognize, however, that amplification can be accomplished by any known method, such as ligase chain reaction (LCR), Qβ-replicase amplification, rolling circle amplification, transcription amplification, self-sustained sequence replication, nucleic acid sequence-based amplification (NASBA), each of which provides sufficient amplification. Branched-DNA technology may also be used to qualitatively or quantitatively determine the amount of nucleic acid encoding LKB1 is present in a particular sample. Nolte reviews branched-DNA signal amplification for direct quantitation of nucleic acid sequences in clinical samples (Nolte, 1998, Adv. Clin. Chem. 33:201-235).

The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; U.S. Pat. No. 4,683,202 (Mullis); which are incorporated herein by reference in their entirety. PCR reagents and protocols are also available from commercial vendors, such as Roche Molecular Systems. PCR may be carried out as an automated process with a thermostable enzyme. In this process, the temperature of the reaction mixture is cycled through a denaturing region, a primer annealing region, and an extension reaction region automatically. Machines specifically adapted for this purpose are commercially available.

5.3.2. High Throughput and Single Molecule Sequencing Technology

Suitable next generation sequencing technologies are widely available. Examples include the 454 Life Sciences platform (Roche, Branford, Conn.) (Margulies et al. 2005 Nature, 437, 376-380); DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453 (Barany et al.); or the Helicos True Single Molecule DNA sequencing technology (Harris et al., 2008 Science, 320, 106-109; U.S. Pat. Nos. 7,037,687 and 7,645,596 (Williams et al.); U.S. Pat. No. 7,169,560 (Lapidus et al.); U.S. Pat. No. 7,769,400 (Harris)), the single molecule, real-time (SMRT™) technology of Pacific Biosciences, and sequencing (Soni and Meller, 2007, Clin. Chem. 53, 1996-2001) which are incorporated herein by reference in their entirety. These systems allow the sequencing of many nucleic acid molecules isolated from a specimen at high orders of multiplexing in a parallel fashion (Dear, 2003, Brief Funct. Genomic Proteomic, 1(4), 397-416 and McCaughan and Dear, 2010, J. Pathol., 220, 297-306). Each of these platforms allows sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination. Machines and reagents for pyrosequencing are available from Qiagen, Inc. (Valencia, Calif.). An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano el al., 2003, J. Biotech. 102, 117-124). Such a system can be used to exponentially amplify amplification products generated by a process described herein, e.g., by ligating a heterologous nucleic acid to the first amplification product generated by a process described herein.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing or detection, energy is transferred between two fluorescent dyes, sometimes polymethine cyanine dyes Cy3 and Cy5, through long-range dipole interactions. The donor is excited at its specific excitation wavelength and the excited slate energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each other for energy transfer to occur successfully.

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., Braslavsky et al., PNAS 100(7): 3960-3964 (2003); U.S. Pat. No. 7,297,518 (Quake et al.) which are incorporated herein by reference in their entirety). Such a system can be used to directly sequence amplification products generated by processes described herein. In some embodiments the released linear amplification product can be hybridized to a primer that contains sequences complementary to immobilized capture sequences present on a solid support, a bead or glass slide for example. Hybridization of the primer-released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

The technology may be practiced with digital PCR. Digital PCR was developed by Kalinina and colleagues (Kalinina et al, 1997, Nucleic Acids Res. 25; 1999-2004) and further developed by Vogelstein and Kinzler (1999, Proc. Natl. Acad. Sci, U.S.A. 96; 9236-9241). The application of digital PCR is described by Cantor et al. (PCT Pub. Nos. WO 2005/023091A2 (Cantor et al.); WO 2007/092473 A2, (Quake et al.)), which are hereby incorporated by reference in their entirety. Digital PCR takes advantage of nucleic acid (DNA, cDNA or RNA) amplification on a single molecule level, and offers a highly sensitive method for quantifying low copy number nucleic acid. Fluidigm® Corporation offers systems for the digital analysis of nucleic acids.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in PCT Pub. No. WO 2009/091934 (Cantor).

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected.

A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

The invention encompasses any method known in the art for enhancing the sensitivity of the detectable signal in such assays, including, but not limited to, the use of cyclic probe technology (Bakkaoui et al., 1996, BioTechniques 20: 240-8, which is incorporated herein by reference in its entirety); and the use of branched probes (Urdea et al 1993, Clin. Chem. 39, 725-6; which is incorporated herein by reference in its entirety). The hybridization complexes are detected according to well-known techniques in the art.

Reverse transcribed or amplified nucleic acids may be modified nucleic acids. Modified nucleic acids can include nucleotide analogs, and in certain embodiments include a detectable label and/or a capture agent. Examples of detectable labels include, without limitation, fluorophores, radioisotopes, colorimetric agents, light emitting agents, chemiluminescent agents, light scattering agents, enzymes and the like. Examples of capture agents include, without limitation, an agent from a binding pair selected from antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) pairs, and the like. Modified nucleic acids having a capture agent can be immobilized to a solid support in certain embodiments.

5.4. Antibody Staining/Detection

In some embodiments, the invention may encompass detecting and/or quantitating using antibodies either alone or in conjunction with other tests. Antibody reagents can be used in assays to detect LKB1 expression levels in patient samples using any of a number of immunoassays known to those skilled in the art. Immunoassay techniques and protocols are generally described in Price and Newman, “Principles and Practice of Immunoassay.” 2nd Edition, Grove's Dictionaries, 1997; and Gosling, “Immunoassays: A Practical Approach.” Oxford University Press, 2000. A variety of immunoassay techniques, including competitive and non-competitive immunoassays, can be used. See, e.g., Self et al., 1996, Curr. Opin. Biotechnol., 7, 60-65. The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., 1997, Electrophoresis, 18, 2184-2193; Bao, 1997, J. Chromatogr. B. Biomed. Sci., 699, 463-480. Liposome immunoassays, such as flow-injection liposome immunoassays and liposome immunosensors, are also suitable for use in the present invention. See, e.g., Rongen et al., 1997, J. Immunol. Methods, 204, 105-133. In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.) and can be performed using a Behring Nephelometer Analyzer (Fink et al., 1989, J. Clin. Chem. Clin. Biochem., 27, 261-276).

Specific immunological binding of the antibody to nucleic acids can be detected directly or indirectly. Direct labels include fluorescent or luminescent tags, metals, dyes, radionuclides, and the like, attached to the antibody. An antibody labeled with iodine-125 ¹²⁵I can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoeiythrin, R-phycoeiythrin, rhodamine, Texas red, and lissamine. Indirect labels include various enzymes well known in the art, such as horseradish peroxidase (IIRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-/3-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support This strip can then be dipped into the test sample and processed quickly through washes and detection steps to generate a measurable signal, such as a colored spot. The antibodies may be in an array one or more antibodies, single or double stranded nucleic acids, proteins, peptides or fragments thereof, amino acid probes, or phage display libraries. Many protein/antibody arrays are described in the art These include, for example, arrays produced by Ciphergen Biosystems (Fremont, Calif.), Packard Bioscience Company (Meriden Conn.), Zyomyx (Hayward, Calif.) and Phylos (Lexington, Mass.). Examples of such arrays are described in the following patents: U.S. Pat. No. 6,225,047 (Hutchens and Yip); U.S. Pat. No. 6,537,749 (Kuimelis and Wagner); and U.S. Pat. No. 6,329,209 (Wagner et al.), all of which are incorporated herein by reference in their entirety.

In alternative embodiments, the invention encompasses use of additional LKB1 specific gene expression and/or antibody assays either in situ, i.e., directly upon tissue sections (fixed and/or frozen) of patient tissue obtained from biopsies or resections, such that no nucleic acid purification is necessary; or based on extracted and/or amplified nucleic acids. For in situ procedures see, e.g., Nuovo, G. J., 1992, PCR In Situ Hybridization: Protocols And Applications. Raven Press, NY, which is incorporated herein by reference in its entirety.

Methods for making nucleic acid microarrays are known to the skilled artisan and are described, for example, in Lockhart et al., 1996, Nat. Biotech. 14, 1675-1680, 1996 Schena et al., 1996, Proc. Natl. Acad. Sci. USA, 93, 10614-10619, U.S. Pat. No. 5,837,832 (Chee et al.) and PCT Pub. No. WO 00/56934 (Englert et al.), herein incorporated by reference. To produce a nucleic acid microarray, oligonucleotides may be synthesized or bound to the surface of a substrate using a chemical coupling procedure and an ink jet application apparatus, as described U.S. Pat. No. 6,015,880 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

The measurement of LKB1 may alone, or in conjunction with other tools discussed above (antibody staining, PCR, CGH, FISH) may have several other non-limiting uses. Amongst these uses are: (i) reclassifying specimens that were indeterminate or difficult to identify in a pathology laboratory; (ii) deciding to follow up with additional tests or more invasive examination such as a laproscopy; (iii) determining the frequency of follow up visits; (iv) initiating other investigatory analysis such as a blood draws or other biopsies; or (v) determine subjects suitable for particular in vitro fertilization (TVF) protocols.

5.5. Compositions and Kits

The invention provides compositions and kits measuring LKB1 polypeptides or polynucleotides described herein using antibodies specific for the polypeptides or nucleic acids specific for the polynucleotides. Kits for carrying put the diagnostic assays of the invention typically include, in suitable container means, (i) a probe that comprises an antibody or nucleic acid sequence that specifically binds to the marker polypeptides or polynucleotides of the invention, (ii) a label for detecting the presence of the probe, and (iii) instructions for how to measure the level of expression (or polypeptide or polynucleotide). The kits may include several antibodies or polynucleotide sequences encoding polypeptides of the invention, e.g., a first antibody and/or second and/or third and/or additional antibodies that recognize a protein encoded by a LKB1 gene. The container means of the kits will generally include at least one vial, test tube, flask, bottle, syringe and/or other container into which a first antibody specific for one of the polypeptides or a first nucleic acid specific for one of the polynucleotides of the present invention may be placed and/or suitably aliquoted. Where a second and/or third and/or additional component is provided, the kit will also generally contain a second, third and/or other additional container into which this component may be placed. Alternatively, a container may contain a mixture of more than one antibody or nucleic acid reagent, each reagent specifically binding a different marker in accordance with the present invention. The kits of the present invention will also typically include means for containing the antibody or nucleic acid probes in close confinement for commercial sale. Such containers may include injection and/or blow-molded plastic containers into which the desired vials are retained.

The kits may further comprise positive and negative controls, as well as instructions for the use of kit components contained therein, in accordance with the methods of the present invention.

5.6. In Vivo Imaging

The various markers of the invention also provide reagents for in vivo imaging such as, for instance, the imaging of LKB1 using labeled reagents that detect (i) LKB1 nucleic acids, (ii) a LKB1 polypeptide or polynucleotide. For in vivo imaging purposes, reagents that detect the presence of these proteins or genes, such as antibodies, may be labeled with a positron-emitting isotope (e.g., 18F) for positron emission tomography (PET), gamma-ray isotope (e.g., 99mTc) for single photon emission computed tomography (SPECT), a paramagnetic molecule or nanoparticle (e.g., Gd³⁺ chelate or coated magnetite nanoparticle) for magnetic resonance imaging (MRI), a near-infrared fluorophore for near-infra red (near-IR) imaging, a luciferase (firefly, bacterial, or coelenterate), green fluorescent protein, or other luminescent molecule for bioluminescence imaging, or a perfluorocarbon-filled vesicle for ultrasound. Fluorodeoxyglucose (FDG)-PET metabolic uptake alone or in combination with MRI is particularly useful.

Unless defined otherwise, all technical and scientific terms used herein have the same meaning as commonly understood by one of ordinary skill in the art to which this invention belongs. The article “a” and “an” are used herein to refer to one or more than one (i.e., to at least one) of the grammatical objects) of the article. By way of example, “an element” means one or more elements.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The following Examples further illustrate the invention and are not intended to limit the scope of the invention. In particular, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### 6. EXAMPLES

6.1. Summary

Methods Patients treated at the University of North Carolina from 1990 to 2009 with NSCLC provided frozen, surgically extracted tumors for analysis. LKB1 and KRAS GE were measured using Agilent 44,000 feature custom-designed arrays. CN were assessed by Affymetrix GeneChip Human Mapping 250K Sty Array or the Genome-Wide Human SNP Array 6.0. Gene mutation was detected using exon sequencing technology. Associations between different measurements of genetic alterations were tested. Integrated analysis was conducted to assess the relationship between these genetic markers and brain metastasis. A predictive model was proposed to use generic measurement for brain metastasis prediction.

Findings 17 of 193 patients developed brain metastasis during the course of the disease. LKB1 wild type tumors had significantly higher LKB1 CN (p=0.005) and GE (p=0.011) than the LKB1 mutant group. KRAS wild type tumors had significantly lower KRAS GE (p=0.002) and lower CN, although the latter failed to be significant (p=0.863). Lower LKB1 CN and KRAS mutation were significantly associated with more brain metastasis, after controlling for nodal (N) stage and patient age. An ROC curve was plotted to estimate the performance of this prediction model with an area under the curve (AUC) of 0.852 showing this multivariate model to be robust.

Interpretation LKB1 CN can be used as a preliminary genetic marker to predict brain metastasis in NSCLC. Further validation studies are needed to evaluate the strength of association.

In this study, we seek to identify how LKB1 alteration, assessed by gene mutation, gene expression (GE) and copy number (CN) change, can predict brain metastasis in a group of NSCLC patients in conjunction with KRAS aberration, which has been shown to have a synergistic effect with LKB1 inactivation in lung cancer development and metastasis ⁶.

Method

Tumor Collection

Frozen tumors were collected from patients who received curative surgery at the University of North Carolina (UNC) with NSCLC diagnosis from December 1990 to September 2009. Tissues were flash-frozen and stored at −80° C. until time of analysis. Tumor histology includes adenocarcinoma, adenosquamous carcinoma, bronchiolo-alveolar carcinoma, large cell carcinoma and squamous cell carcinoma. Medical record related to diagnosis and metastasis was annotated from patients' charts. Patients were followed up until January, 2011 for vital and recurrence information. Brain metastasis was confirmed by diagnostic imaging. The study was approved by Institutional Review Board (IRB) under protocols 90-0573 and 07-0120.

GE Microarray

GE was measured by Agilent 44 K microarrays (human tumor). Total RNA from tumor tissues was isolated using the RNeasy kit following the manufacturer's protocols (Qiagen, Valencia, Calif., USA). Total RNA-lug was converted to labeled cRNA with nucleotides coupled to a fluorescent dye (Cy3) using the Quick Amp Kit (Agilent Technologies, Palo Alto, Calif.). Universal RNA from Invitrogen was labeled with Cy5 as a reference. Samples were purified using an RNeasy kit (Qiagen) and quantified for dye integration using a Nanodrop-8000 (Thermo Scientific). Following quantification, samples were hybridized overnight in a rotating hybridization oven and washed/scanned using an Agilent scanner. Microarrays were processed by normexp background correction and loess normalization ¹⁰.

LKB1 and KRAS Mutations

Genomic DNA was extracted from tumor tissues using Qiagen QiaAmp DNA kit and sent to Polymorphic DNA Technologies Inc. (Almeda Calif.) for direct exon sequencing on ABI 3730XL DNA sequencers to detect LKB1 and KRAS mutations. Regions of LKB1 and KRAS sequencing were described elsewhere¹¹, with all nine exons of LKB1 and exon 2 of KRAS, which harbors more than 95% of KRAS mutation sequenced. Non-synonymous or splice site differences compared to reference sequence were considered as mutations.

LKB1 and KRAS CN Assessment

CN microarray of tumor DNA was performed using the Affymetrix GeneChip Human Mapping 250K Sty Array or the Genome-Wide Human SNP Array 6.0 (Affymetrix, Inc., Santa Clara, Calif.) according to the manufacturer's instructions. CN for each marker was calculated using CRMA_v2 ¹², which performs log2 transformation on preprocessed signal intensity. CN for each marker was taken to be log2 (tumor sample/normal estimate), where the normal estimate was calculated using the mean intensity from all normal specimens. CN for LKB1 and KRAS in each sample were taken as the median values of estimated copy numbers across all markers that are within the 100 kb region upstream or downstream of the genes.

Statistical Analysis

All statistical analysis was performed using R 2.10.1 software (http://cran.r-project.org) unless otherwise stated. Patients follow up lime was calculated using “reverse” Kaplan-Meier analysis in

which the outcomes ‘dead’ and ‘censored’ are exchanged¹³. Pairwise association between patients' baseline characteristics, including gender, race, stage, tumor histology and smoking status, and genetic biomarkers, including LKB1 and KRAS mutation, GE and CN, were tested using Fisher exact test for categorical variables and two sample t-test for continuous variables. Logistic regression was used to test the association between each of the variables and brain metastasis. Variables showed significant association with brain metastasis at the 0.1 level in univariate analysis were considered for inclusion in multivariate analysis. For all the analyses, a complete case approach was used to handle missing data. All statistical tests were two sided tests and all reported confidence intervals were constructed at a two sided 95% confidence level.

Results

Patient Characteristics with Respect to Genetic Biomarkers

193 of the patients provided sufficient tissue for at least one measurement of LKB1 alteration, either by gene expression microarray (182 samples), CN (172 samples) or gene sequencing (179 samples) and were included in subsequent analysis. Diagnosis age ranges from 41 to 90 with a median of 66 years; approximately half of these patients (95) are males and most of them (177) had smoking history. The majority of these patients (172) were diagnosed when the tumor was still small (T1 or T2). About half of the patients (100) had adenocarcinoma, and most of the others had squamous cell carcinoma (64) or adenosquamous carcinoma (11). The median follow up time was 89 months. Only 20 patients were lost to follow up before 60 months, with a median follow up time 46 months. The median survival time of these 193 patients was 44 months (CI 36-62 months). 17 (-10%) patients had brain recurrence with a median survival time after brain metastasis as 8 months (CI: 5.3-37.6 months).

Table 1 summarized how patient characteristics associated with genetic biomarkers LKB1 and KRAS. Overall, 11 of 179 samples (6.39%) that were sequenced for LKB1 had non-synonymous or splice site mutation; 23 of the 187 samples (12.3%) that were sequenced for KRAS reported non-synonymous KRAS mutation. The overall mutation rate is lower than previously reported in Caucasian population^{4,14} but comparable to the reported mutation rate in the East Asian population^{15.} Consistent with previous research^{4,14,} LKB1 mutations were more common in adenocarcinoma (8/91) than in non-adenocarcinoma (3/87), although the difference failed to be significant (p=0.21). Similarly, KRAS mutations were more frequent in adenocarcinoma (21/98) than other tumor histology (2/89, p=0.001). Meanwhile, adenocarcinomas had significantly lower LKB1 expression (p=0.001), lower LKB1 CN (p=0.044) and higher KRAS expression (p=0.035) compared to the non-adenocarcinoma group. Smoking was related to LKB1 and KRAS mutation¹⁴; all samples that were mutant for LKB1 were smokers and only one KRAS mutant was a non-smoker, although the association was not significant. Both LKB1 and KRAS mutation were associated with earlier T stage, with none of the LKB1 mutant samples and only one KRAS mutation samples having stage T3 or T4. Gender and race were not associated with LKB1 or KRAS measurement.

Associations Between Genetic Markers

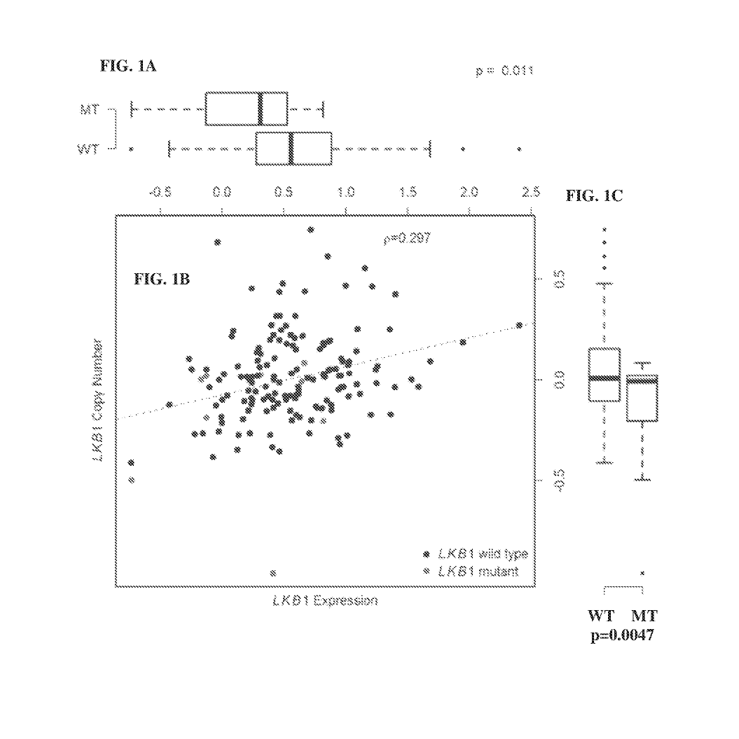

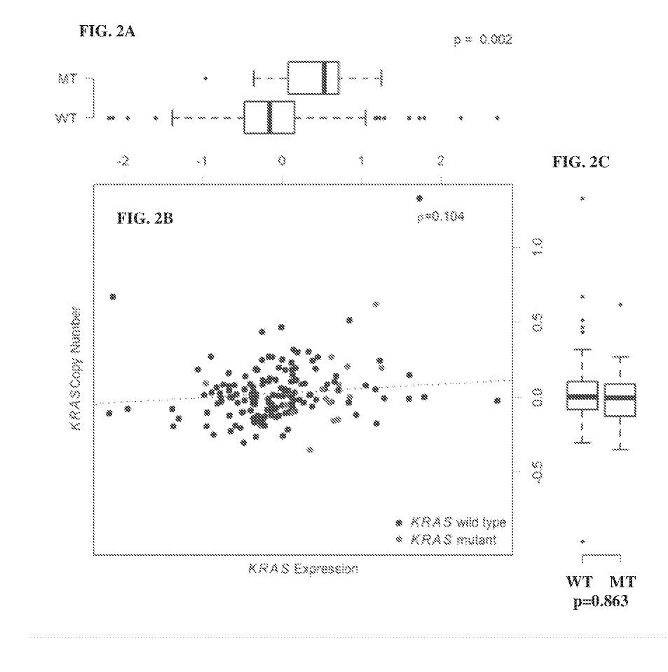

Reduced expression, decreased CN and gene mutation are three major ways of gene silencing. The correlation between these genetic measurements for LKB1 and KRAS was captured in FIG. 1 and FIG. 2. LKB1 mutation was significantly associated with lower GE (FIG. 1A, p=0.011) and lower CN (FIG. 1C, p=0.0047). On the contrary, KRAS mutation was associated with higher expression (FIG. 2A, p=0.002). There is no significant association between KRAS mutation and KRASCN (FIG. 2C). CN and GE are positively correlated in both LKB1 and KRAS, although the association failed to be significant (FIG. 1B, 2B, p=0.297 and 0.104 respectively).

Univariate Association of Patient Characteristics and Genetic Markers with Brain Metastasis Status

17 of the patients had brain metastasis during the follow up. Patients' characteristics with respect to brain metastasis were summarized in Table 2. Neither gender nor race was associated with brain recurrence. All but one patient with brain metastasis were current/former smokers.

Of all patients with brain recurrence, only one had late T (T3 or T4) stage at diagnosis. However, the association failed to be significant because of the small number of brain recurrence. N stage was significantly associated with brain metastasis: the odds ratio of brain recurrence between patients diagnosed with late N stage (N1 or above) and patients with no nodal spread at diagnosis is 4.4(CI: 1.55-13.65). Brain recurrence in adenocarcinoma (12/100) is more frequent than in non-adenocarcinoma (5/93), although the association failed to be significant (p=0.135). Of the genetic markers, KRAS mutation was significantly associated with brain metastasis (p=0.005). LKB1 CN was borderline significantly associated with brain metastasis (p=0.069). Higher LKB1 expression and 1KB1 wild type were also associated with less brain metastasis, although the result did not achieve statistical significance.

Multivariate Association of Patient Characteristics and Genetic Markers with Brain Metastasis Status

Variables that were associated with brain recurrence with p value<0.1 in univariate models were considered for inclusion in the multivariate model (Table 3). LKB1 CN and KRAS mutation were significantly associated with brain recurrence after adjusting for patient age and nodal status. Patients with higher LKB1 CN or wild type KRAS had lower risk of developing brain recurrence. The odds of brain metastasis in mutant KRAS patients were estimated to be 7.38 (CI 1.93-28.7) times higher than the odds of brain recurrence in patients with wild type KRAS, after adjusting for age, KRAS mutation status and N stage. The odds of brain recurrence in patients with one decrease of LKB1 CN were −22 times higher than that in LKB1 copy neutral patients, after controlling for age, KRAS mutation and N stage.

Model Predictions of Brain Metastasis

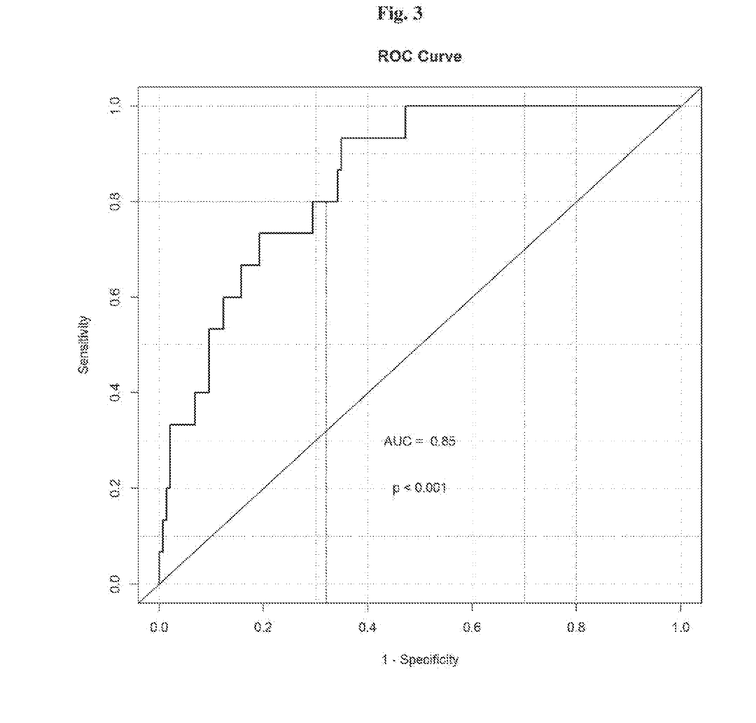

ROC curve was used to evaluate the prediction of the previous multivariate model (FIG. 3). The area under the curve (AUC) was estimated to be 0.850 and significantly different from 0.5 (p=0.001). With a false positive rate of approximately 30%, the model successfully captured 80% of the true brain metastasis patients.

Discussion

The high morbidity and mortality of lung cancer is greatly attributable to the poor understanding of biochemical pathways that are involved in cancer progression and metastasis. Identifying patients at higher risk of brain metastasis by genetic markers would help with earlier diagnosis and better treatment choice. Although the prognosis of NSCLC with brain metastasis has been poor, recent clinical studies have suggested promising prevention procedures to reduce brain metastasis as well as treatment strategies that can prolong patients' survival. Prophylactic cranial irradiation (PCI) has been shown to decrease the incidence of intracranial metastasis in NSCLC ¹⁶, although a survival benefit has not been established. This lack of survival benefit might be related to the fact that patients received PCI without consideration of their initial risk of developing brain metastasis.

On the treatment side, combination of surgery, whole brain radiation and stereotactic radiosurgery are common treatment options for brain metastasis. Active treatment, especially chemotherapy has been reported to be effective in small cell lung carcinomas (SCLC) with brain metastasis ¹⁷. In NSCLC, both whole brain radiotherapy (WBRT) and local treatment (surgery or radiosurgery) are the cornerstones of treatment. When metastases are few and resectable, surgery or stereotactic radiosurgery with WBRT at recurrence are shown to convey significant survival benefit ¹⁸. On the other hand, brain radiation is associated with patient morbidity and impaired quality of life, depending on radiation dose and type ¹⁹. Thus, a set of prognostic markers, even if they do not have 100% specificity and sensitivity, would be very helpful in determining treatment strategies for patients with risk of developing brain metastasis. In this study, we conducted a comprehensive assessment of a tumor suppressor gene, LKB1, and a related oncogene, KRAS, with relation to brain metastasis and suggested that LKB1 CN alteration can be a key component in predicting brain metastasis in NSCLC.

LKB1 is one of the most important tumor suppressor genes and is observed to be inactivated in approximately 30% of all NSCLCs ⁴. Studies to explore its inactivation mechanisms as well as subsequent signaling pathway change are therefore warranted. LKB1 encodes a widely expressed serine/threonine protein kinase whose primary action is through 5′-AMP-activated protein kinase (AMPK) to regulate metabolism and ensure efficient energy production with minimal waste in times of stress²⁰. AMPK's control of cell proliferation is mainly through the mammalian target of rapamycin (mTOR) kinase, which regulates numerous downstream targets, such as amino acid transporters, VEGF, p70 ribosomal protein S6 kinase (S6K)²¹. Also, AMPK phosphorylates TSC2 activating the TSC1-2 complex, which inhibits RAS homologue enriched in the brain and prevents the activation of mTOR²². LKB1 loss impairs downstream signaling of AMPK, leading to unsuppressed cell proliferation ²³LKB1 deficiency can be associated with increased expression of genes believed to control angiogenesis and metastatic potentials ⁹.

LKB1 can be inactivated through a variety of mechanisms, including a combination of gene mutation, deletion and epigenetic events, like prompter methylation. Somatic mutations, mainly nonsense or frame-shift mutations, can result in truncated and dysfunctional proteins ²⁴. Somatic mutation can account for only a small fraction of tumors and cannot be the sole reason of LKB1 in activation⁵. Promoter methylation resulting in reduced expression was shown to account for a small percentage of depressed LKB1 expression as well ²⁵. Gene deletion is a frequent mechanism of LKB1 loss, which can be assessed by CN²⁶. The fact that the LKB1 mutant group also had lower CN is consistent with the common two-hit model for tumorigenesis which requires an individual heterozygous for a mutant tumor suppressor gene to lose the normal allele in order for tumor development, which is frequently achieved through deletion of the normal allele. Based on clear evidence in animal models that LKB1 haploinsufficiency accelerates KRAS driven lung cancer in mice ⁶, even a single copy inactivation of LKB1 might be oncogenic. A recent report using murine melanocytes models showed that somatically inactivating LKB1 with KRAS activation can induce highly metastatic melanoma with 100% penetrance, suggesting that LKB1 inactivation can greatly facilitates metastasis, especially in the context of RAS activation ²⁷. CN might be a good proxy for mutation for LKB1, supported by our result that the mutated group is associated with reduced CN. It is also possible that a combination of these events is at work in inducing cancers and tumor invasion. Finding the best measurement that can adequately predict, brain metastasis and is relatively straightforward to estimate in the clinical setting is very helpful in patient management.

The development of cancer is a complex process which requires the accumulation of damage to the cell's growth-controlling genes, including damage to tumor suppressor genes and proto-oncogenes. Most carcinomas are initiated by the loss of function of a tumor suppressor gene, followed by alterations in oncogenes and additional tumor suppressor genes. Usually considered to be recessive in causing cancer, tumor suppressor gene alteration is more difficult to evaluate than oncogene changes. Gene mutations are difficult to evaluate in clinical settings due to DNA fragmentation ²⁸ and relatively high prices for gene sequencing, especially for large genes. GE measurement from paraffin embedded samples can suffer from RNA degradation, cost of microarray, and limited access to tissues and lack of rigorous standards for data collection, analysis arid validation ²⁹. Measurement of CN is relatively easy in clinical settings by in situ hybridization ³⁰.

The current study has inherent limitations. Most notably, the number of brain metastasis patients included in this study is relatively small (17 patients), which inhibits further validation of our predictive model. The estimated odds ratio should be used as an indication of association direction, rather than being a concrete measurement of genetic effect. The aim of this study is to find clinical relevant markers which can help with patient management, instead of evaluating the mechanism by which LKB1 is involved in NSCLC brain metastasis. On the other hand, the hypothesis of this study is driven by clinical expertise before any data were collected. Because of the malignancy of brain metastasis and the available beneficial treatment, a reasonable prediction model, even though not perfect, can help the decision making process in patient management. A strong association between KRAS mutation and brain metastasis was also observed in our study. Given the dramatic increase in tumor incidence and metastasis in the KRAS, LKB1^{lox/lox} in mouse model reported in previous investigation ^{6,} this also suggested that Ras-dependent signals and LKB1 loss might display a specific synergy in tumor development and metastasis. Unfortunately, the sample size is insufficient to assess the interaction between LKB1 and KRAS alteration.

In conclusion, we have investigated how different measurement of LKB1 and KRAS alteration can affect the possibility of having brain metastasis in NSCLC. The predictive model can be useful in selecting patients at higher risk of brain metastasis and eligible for corresponding prevention and treatment in clinical setting. Further study is needed for validation of our predictive model as well as on the mechanism by which LKB1 and KRAS can be associated with brain metastasis in lung cancers.

TABLE 1
Patient characteristics by genetic biomarkers

| | LKB1 mutation | | Kras mutation | | LKB1 | LkB1 copy^{φ} |
|---|---|---|---|---|---|---|
| | status | | status | | expression | number |
| | WT | MT | WT | MT | mean | mean |
| # of Samples | 167 | 11 | 164 | 23 | | |
| Female | 90 | 3 | 87 | 10 | 0.506 | −0.0002 |
| Male | 77 | 8 | 77 | 13 | 0.582 | 0.007 |
| White^{ζ} | 135 | 9 | 131 | 20 | 0.549 | 0.003 |
| Black | 28 | 2 | 29 | 3 | 0.533 | 0.023 |
| Current/Former Smoker^{ζ} | 151 | 11 | 149 | 22 | 0.542 | −0.0007 |
| Never/Light smoker | 15 | 0 | 14 | 1 | 0.647 | 0.064 |
| Adenocarcinoma^{ζ} | 83 | 8 | 77** | 21** | 0.408** | −0.031* |
| Non-adenocarcinoma | 84 | 3 | 87** | 2** | 0.698** | 0.039* |
| T stage^{ζ} | | | | | | |
| T1-T2 | 150 | 11 | 145 | 22 | 0.546 | −0.0001 |
| T3-T4 | 15 | 0 | 17 | 1 | 0.5645 | 0.015 |
| N stage^{ζ} | | | | | | |
| N0 | 111 | 9 | 113 | 12 | 0.571 | 0.002 |
| N1 or above | 48 | 2 | 45 | 9 | 0.5115 | −0.0008 |

**p < 0.001
*p < 0.05
^{ζ}number doesn't sum to total because of missing values

TABLE 2
patient characteristics and genetic marker by disease outcome

| | brain | | no brain | | | |
|---|---|---|---|---|---|---|
| | recurrence | | recurrence | | Odds Ratio ^{φ} | P |
| | (Column %) | | (Column %) | | (95% CI) | values |
| # of patients | 17 | | 176 | | | |
| Age at Diagnosis(mean) | 58.26 | | 66.06 | | 0.9379 | |
| | | | | | (0.79-0.98) | 0.0082 |
| Gender | | | | | | |
| Female | 10 | (58.8) | 88 | (50) | 1.429 | |
| Male | 7 | (41.2) | 88 | (50) | (0.52, 4.09) | 0.489 |
| Race | | | | | | |
| White ^{ζ} | 13 | (76.5) | 142 | (82.6) | 0.686 | |
| Black | 4 | (23.5) | 30 | (17.4) | (0.225, 2.57) | 0.535 |
| Smoking status ^{ζ} | | | | | | |
| Current/Former Smoker ^{ζ} | 16 | (94.1) | 161 | (92) | 1.39 | |
| Never/Light Smoker | 1 | (5.9) | 14 | (8) | (0.253, 26.02) | 0.757 |
| Tumor histology ^{ζ} | | | | | | |
| Adenocarcinoma | 12 | (70.6) | 88 | (50) | 2.40 | |
| Non-adenocarcinoma | 5 | (29.4) | 88 | (50) | (0.851, 7.80) | 0.135 |
| T stages ^{ζ} | | | | | | |
| T3-T4 | 1 | (6.25) | 18 | (10.3) | 0.581 | |
| T0-T1-T2 | 15 | (93.75) | 157 | (89.7) | (0.031-3.14) | 0.609 |
| N stages ^{ζ} | | | | | | |
| N1 or above | 10 | (62.5) | 46 | (27.4) | 4.42 | |
| N0 | 6 | (37.5) | 122 | (72.6) | (1.55, 13.65) | 0.0063 |
| LKB1 mutation ^{ζ} | | | | | | |
| Mutant | 2 | (12.5) | 9 | (5.56) | 2.43 | |
| Wild type | 14 | (87.5) | 153 | (94.4) | (0.35-10.65) | 0.285 |
| LKB1 expression ^{ζ} | 0.472 | | 0.555 | | 0.689 | |
| mean | | | | | (0.223, 2.01) | 0.507 |
| LKB1 copy number ^{ζ} | −0.0947 | | 0.0133 | | 0.111 | |
| (mean) | | | | | (0.0089, 1.14) | 0.069 |
| KRAS mutation ^{ζ} | | | | | | |
| Mutant | 6 | (35.3) | 17 | (10) | 4.909 | |
| Wild type | 11 | (64.7) | 153 | (90) | (1.531, 14.71) | 0.0051 |

| | | | | |
|---|---|---|---|---|
| KRAS expression ^{ζ} | −0.071 | −0.034 | 0.930 | |
| mean | | | (0.441, 1.882) | 0.844 |
| KRAS copy number ^{ζ} | 0.0062 | 0.0275 | 0.5835 | |
| mean | | | (0.039, 6.572) | 0.689 |

*p < 0.05
** p < 0.001
^{ζ} number doesn't sum to total because of missing values
^{φ} For each variable, the reported value is the odds ratio of brain metastasis in patients with characteristics in the first row compared to the patients with characterisitics in the second row. For example, for gender, the odds of having brain recurrence in females are 1.429 times the odds of brain metastasis in males.
P values were calculated as follows: for continuous variables, two sample t-test was used to compare the mean in the brain metastasis group and the non-brain metastasis group. For categorical variables, fisher exact test was used to compare the proportion of brain metastasis in each group. Odds ratio and confidence intervals were generated by fitting logistic models using each of the variables with brain recurrence as response variable.

TABLE 3
Multivariate association between genetic
biomarkers and disease outcome

| | Odds Ratio | 95% CI | P values |
|---|---|---|---|
| Age at diagnosis | 0.940 | (0.886, 0.993) | 0.032 |
| LKB1 copy number | 22.78 | (1.32, 461, ) | 0.032 |
| KRAS mutation | 7.380 | (1.926, 28.68) | 0.003 |
| N stage | 2.780 | (0.813, 9.923) | 0.103 |

### REFERENCES

1. Govindan R, Page N, Morgensztern D, et al. Changing epidemiology of small-cell lung cancer in the United States over the last 30 years: analysis of the surveillance, epidemiologic, and end results database. J Clin Oncol 2006; 24(28):4539-44.
2. Gavrilovic I T, Posner J B. Brain metastases: epidemiology and pathophysiology. J Neurooncol 2005; 75(1):5-14.
3. Ampil F, Caldito G, Milligan S, Mills G, Nanda A. The elderly with synchronous non-small cell lung cancer and solitary brain metastasis: does palliative thoracic radiotherapy have a useful role? Lung Cancer 2007; 57(1):60-5.
4. Sanchez-Cespedes M, Parrella P, Esteller M, et al. Inactivation of LKB1/STK11 is a common event in adenocarcinomas of the lung. Cancer Res 2002; 62(13):3659-62.
5. Matsumoto S, Iwakawa R, Takahashi K, et al. Prevalence and specificity of LKB1 genetic alterations in lung cancers. Oncogene 2007; 26(40):5911-8.
6. Ji H, Ramsey M R, Hayes D N, et al. LKB1 modulates lung cancer differentiation and metastasis. Nature 2007; 448(7155):807-10.
7. Hemminki A, Markie D, Tomlinson I, et al. A serine/threonine kinase gene defective in Peutz-Jeghers syndrome. Nature 1998; 391(6663):184-7.
8. Sobottka S B, Haase M, Fitze G, Hahn M, Schackert H K, Schackert G. Frequent loss of heterozygosity at the 19p13.3 locus without LKB1/STK11 mutations in human carcinoma metastases to the brain. J Neurooncol 2000; 49(3):187-95.
9. Zhuang Z G, Di G H, Shen Z Z, Ding J, Shao Z M. Enhanced expression of LKB1 in breast cancer cells attenuates angiogenesis, invasion, and metastatic potential. Mol Cancer Res 2006; 4(11):843-9.
10. Ritchie M E, Silver J, Oshlack A, et al. A comparison of background correction methods for two-colour microarrays. Bioinformatics 2007; 23(20):2700-7.
11. Wilkerson M D, Yin X, Walter V, et al. Differential pathogenesis of lung adenocarcinoma subtypes involving sequence mutations, copy number, chromosomal instability, and methylation. PLoS One 2012; 7(5):e36530.
12. Bengtsson H, Ray A, Spellman P, Speed T P. A single-sample method for normalizing and combining full-resolution copy numbers from multiple platforms, labs and analysis methods. Bioinformatics 2009; 25(7):861-7.
13. Shuster J J. Median follow-up in clinical trials. J Clin Oncol 1991; 9(1):191-2.
14. Ding L, Getz G, Wheeler D A, et al. Somatic mutations affect key pathways in lung adenocarcinoma. Nature 2008; 455(7216):1069-75.
15. Koivunen J P, Kim J, Lee J, et al. Mutations in the LKB1 tumour suppressor are frequently detected in tumours from Caucasian but not Asian lung cancer patients. Br J Cancer 2008; 99(2):245-52.
16. Gore E M, Bae K, Wong S J, et al. Phase III comparison of prophylactic cranial irradiation versus observation in patients with locally advanced non-small-cell lung cancer; primary analysis of radiation therapy oncology group study RTOG 0214. J Clin Oncol 2010; 29(3):272-8.
17. Chen G, Huynh M, Chen A, Fehrenbacher L, Gandara D, Lau D. Chemotherapy for brain metastases in small-cell lung cancer. Clin Lung Cancer 2008; 9(l):35-8.
18. Andrews D W, Scott C B, Sperduto P W, et al. Whole brain radiation therapy with or without stereotactic radiosurgery boost for patients with one to three brain metastases: phase III results of the RTOG 9508 randomised trial. Lancet 2004; 363(9422):1665-72.
19. Scoccianti S, Ricardi U. Treatment of brain metastases: Review of phase III randomized controlled trials. Radiother Oncol 2011.
20. William W N, Kim J S, Liu D D, et al. The impact of phosphorylated AMP-activated protein kinase expression on lung cancer survival. Ann Oncol 2011; 23(1):78-85.
21. Makowski L, Hayes D N. Role of LK131 in lung cancer development. Br J Cancer 2008; 99(5):683-8.
22. Garami A, Zwartkruis F J, Nobukuni T, et al. Insulin activation of Rheb, a mediator of mTOR/S6K/4E-B P signaling, is inhibited by TSC1 and 2. Mol Cell 2003; 11(6):1457-66.
23. Carretero J, Medina P P, Blanco R, et al. Dysfunctional AMPK activity, signalling through mTOR and survival in response to energetic stress in LKB1-deficient lung cancer. Oncogene 2007; 26(11):1616-25.
24. Launonen V. Mutations in the human LKB1/STK11 gene. Hum Mutat 2005; 26(4):291-7.
25. Esteller M, Avizienyte E, Corn P G, et al. Epigenetic inactivation of LKB1 in primary tumors associated with the Peutz-Jeghers syndrome. Oncogene 2000; 19(1):164-8.
26. Gill R K, Yang S H, Meerzaman D, et al. Frequent homozygous deletion of the LKB1/STK11 gene in non-small cell lung cancer. Oncogene 2011; 30(35):3784-91.
27. Liu W, Monahan K B, Pfefferle A D, et al. LKB1/STK11 inactivation leads to expansion of a prometastatic tumor subpopulation in melanoma. Cancer Cell 2012; 21(6):751-64.
28. Kotoula V, Charalambous E, Biesmans B, et al. Targeted KRAS mutation assessment on patient tumor histologic material in real time diagnostics. PLoS One 2009; 4(11):e7746.
29. Russo G, Zegar C, Giordano A. Advantages and limitations of microarray technology in human cancer. Oncogene 2003; 22(42):6497-507.
30. Hirsch F R, Varella-Garcia M, Cappuzzo F, et al. Combination of EGFR gene copy number and protein expression predicts outcome for advanced non-small-cell lung cancer patients treated with gefitinib. Ann Oncol 2007; 18(4):752-60.

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

We claim:

1. A method for determining the likelihood of brain metastasis from non-small-cell lung cancer (NSCLC) which comprises:
(a) measuring a level of LKB1 in a sample from the subject; and
(b) if the level of LKB1 is reduced than a normal level, determining that the subject has increased likelihood of brain metastasis.

2. The method of claim 1, wherein the level of LKB1 is measured by copy number.

3. The method of claim 1, wherein the level of LKB1 is measured using a mass spectrometry assay.

4. The method of claim 2, wherein the level of LKB1 is measured using a mass spectrometry assay.

5. The method of claim 1, wherein the sample is a formalin-fixed, paraffin-embedded sample.

6. The method of claim 1, wherein the sample is a fresh-frozen sample.

7. The method of claim 1, wherein the sample is a fresh sample.

8. The method of claim 1, wherein the sample is a primary tumor sample.

9. The method of claim 1, wherein the level of KRAS is also measured.

10. The method of claim 2, wherein the level of KRAS is also measured.

11. A method of selecting non-small-cell lung cancer (NSCLC) patients for prophylactic cranial irradiation (PCI) which comprises measuring the level of LKB1 in a patient sample and if the levels are reduced selecting the patient for prophylactic cranial irradiation (PCI).

12. The method of claim 11, further comprising measuring the level or mutations in KRAS.

13. A kit comprising:
(a) at least one reagent selected from the group consisting of:
(i) a nucleic acid probe capable of specifically hybridizing with an mRNA encoding LKB1;
(ii) a pair of nucleic acid primers capable of PCR amplification of an mRNA encoding LKB1; and
(b) instructions for use in measuring a level of LKB1 in a proliferative phase sample from a subject suspected of having increased likelihood of brain metastasis from NSCLC wherein levels of LKB1 at least 2-fold or greater are indicative of increased likelihood of brain metastasis from NSCLC in the subject.
